(19) United States
(12) Patent Application Publication
(10) Pub. No.: US 2015/0094749 A1
(43) Pub. Date: Apr. 2, 2015

(54) DEVICES, SYSTEMS AND METHODS FOR A PILOTING TIP BUSHING FOR ROTATIONAL ATHERECTOMY

(71) Applicant: Cardiovascular Systems, Inc., St. Paul, MN (US)

(72) Inventors: Nicholas Ellering, Crystal, MN (US); Joseph Higgins, Minnetonka, MN (US)

(21) Appl. No.: 14/567,360

(22) Filed: Dec. 11, 2014

Related U.S. Application Data
(63) Continuation-in-part of application No. 14/535,915, filed on Nov. 7, 2014.
Application No. 14/535,915 is a continuation-in-part of application No. 14/166,207, filed on Jan. 28, 2014.
(60) Provisional application No. 61/782,083, filed on Mar. 14, 2013.

(51) Int. Cl. A61B 17/3207

(52) U.S. Cl. CPC A61B 17/3207; A61B 17/320758; A61B 2017/320741; A61B 2017/320766; A61B 2017/320004
USPC 606/159

(57) ABSTRACT

A high-speed rotational atherectomy device for opening a stenosis in an artery having a given diameter, comprising: a guide wire; a flexible elongated, rotatable drive shaft advanceable over the guide wire, the drive shaft having a proximal end and a distal end; and a piloting element fixedly attached to the drive shaft. When the piloting element is advanced to a stenosis, the piloting element creates a piloting hole when the drive shaft is rotated.

### CROSS-REFERENCE TO RELATED APPLICATIONS

This application is a continuation-in-part of U.S. patent application Ser. No. 14/535,915 filed Nov. 7, 2014, which is a continuation-in-part of U.S. patent application Ser. No. 14/166,207 filed Jan. 28, 2014, which claims priority to U.S. Provisional Application No. 61/782,083, filed Mar. 14, 2013, the entirety of which prior filed applications are hereby incorporated by reference.

### FIELD OF THE INVENTION

The invention relates to devices and methods for removing tissue from body passageways, such as removal of atherosclerotic plaque from arteries, utilizing a high-speed rotational atherectomy device.

### DESCRIPTION OF THE RELATED ART

A variety of techniques and instruments have been developed for use in the removal or repair of tissue in arteries and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patentcy of the opened artery. For non-calcified lesions, balloon angioplasty most often is used alone to open the artery, and stents often are placed to maintain patentcy of the opened artery. Studies have shown, however, that a significant percentage of patients who have undergone balloon angioplasty and had a stent placed in an artery experience stent restenosis—i.e., blockage of the stent which most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patentcy of the artery.

Several kinds of rotational atherectomy devices have been developed for attempting to remove stenotic material. In one type of device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. When rotated at high speeds, the abrasive segment is capable of removing stenotic tissue from an artery. Though this atherectomy device possesses certain advantages over the Auth device due to its flexibility, it also is capable only of opening an artery to a diameter about equal to the diameter of the enlarged abrading surface of the drive shaft since the device is not eccentric in nature.

U.S. Pat. No. 6,494,890 (Shturman) discloses a known atherectomy device having a drive shaft with an enlarged eccentric section, wherein at least a segment of this enlarged section is covered with an abrasive material. When rotated at high speeds, the abrasive segment is capable of removing stenotic tissue from an artery. The device is capable of opening an artery to a diameter that is larger than the resting diameter of the enlarged eccentric section due, in part, to the orbital rotational motion during high speed operation. Since the enlarged eccentric section comprises drive shaft wires that are not bound together, the enlarged eccentric section of the drive shaft may flex during placement within the stenosis or during high speed operation. This flexion allows for a larger diameter opening during high speed operation, but may also provide less control than desired over the diameter of the artery actually abraded. In addition, some stenotic tissue may block the passageway so completely that the Shturman device cannot be placed therethrough. Since Shturman requires that the enlarged eccentric section of the drive shaft be placed within the stenotic tissue to achieve abrasion, it will be less effective in cases where the enlarged eccentric section is prevented from moving into the stenosis. The disclosure of U.S. Pat. No. 6,494,890 is hereby incorporated by reference in its entirety.

U.S. Pat. No. 5,681,336 (Clement) provides a known eccentric tissue removing burr with a coating of abrasive particles secured to a portion of its outer surface by a suitable binding material. This construction is limited, however because, as Clement explains at Col. 3, lines 53-55, that the asymmetrical burr is rotated at “lower speeds than are used with high speed ablation devices, to compensate for heat or imbalance.” That is, given both the size and mass of the solid burr, it is infeasible to rotate the burr at the high speeds used during atherectomy procedures, i.e., 20,000-200,000 rpm. Essentially, the center of mass offset from the rotational axis of the drive shaft would result in development of significant centrifugal force, exerting too much pressure on the wall of the artery and creating too much heat and excessively large particles.

In some situations, at the high rotational speeds of the atherectomy device, when the device is driven into the lesion, it can screw into the lesion. Moreover, the atherectomy device may be limited to a certain size of lesion or stenosis for treatment because of the diameter of the burr. For these and other reasons, it may be desirable to include an abrasive structure positioned distally from the ablation burr to first create a piloting hole in the stenosis before the abrading head contacts the stenosis. Prior art devices, such as U.S. Pat. No. 6,482,216 (Hiblar), have suggested a concentric ablation burr mounted on the driveshaft and a concentric abrasive tip mounted on the end of the guidewire to ablate deposits from the blood vessel or stent without becoming embedded in the deposits as the abrasive tip engages the deposits. However, with such devices, the path and diameter of treatment is limited to the minimum size lesion.

The present invention overcomes these deficiencies and provides, inter alia, the above-referenced improvements.

### BRIEF SUMMARY OF THE INVENTION

The present system is directed in various methods, devices and systems relating to rotational atherectomy. More specifically, a piloting element is mounted on a drive shaft, the piloting element comprising a shape and structure to facilitate opening pilot holes through difficult occlusions and/or stenosis.

In some embodiments, the high-speed rotational atherectomy device for opening a stenosis in an artery having a given diameter, comprises a guide wire having a maximum diameter less than the diameter of the artery; a flexible elongated, rotatable drive shaft advanceable over the guide wire, the drive shaft having a proximal end and a distal end; and a piloting element fixedly attached to the drive shaft proximate a distal end thereof. In some embodiments, the piloting element has a concentric or eccentric profile.

In at least one embodiment, a piloting element comprises a proximal section extending distally from a proximal end of the piloting element, the proximal section having a constant diameter; a distal section extending proximally from a distal end of the piloting element having a diameter at the distal end less than a diameter at the proximal end of the piloting element, the diameter increasing proximally from the distal end; and an intermediate section between the proximal section and the distal section, the intermediate section having a generally parabolic profile, wherein the diameter of the piloting element increases from the constant diameter of the proximal section to a maximum point and then decreases distally towards the distal section. The piloting element can be either concentric or eccentric. In some embodiments, the piloting element has an inner lumen at least at the proximal section with a diameter greater than the diameter of the drive shaft. In at least one embodiment, the piloting element has a diameter less than a diameter of the drive shaft.

A method for opening a stenosis in a blood vessel having a given diameter is also provided, the method comprising: providing a guide wire having a maximum diameter less than the diameter of the artery; advancing the guide wire into a blood vessel to a position proximal to the stenosis; providing a flexible elongated, rotatable drive shaft advanceable over a guide wire, the drive shaft having a maximum diameter less than the diameter of the artery; the drive shaft having a rotational axis; the drive shaft having a piloting element fixedly attached to the drive shaft; advancing the piloting element into the artery to a position proximal to the stenosis; creating a piloting hole by rotating the drive shaft at a sufficient rotational speed. In some embodiments, the piloting element has an orbital path such that the piloting hole has a diameter greater than a maximum diameter of the piloting element.

### BRIEF DESCRIPTION OF THE DRAWINGS

FIG. 1 is a perspective view of a non-limiting exemplary embodiment of a rotational atherectomy device;

FIG. 2 is a perspective view of a non-limiting exemplary embodiment of a piloting element for a rotational atherectomy device;

FIG. 3 is a side view of the piloting element of FIG. 2;

FIG. 4 is an end view of the piloting element of FIGS. 2-3 from a distal end thereof;

FIG. 5 is an end view of the piloting element of FIGS. 2-4 from a proximal end thereof;

FIG. 6 is a perspective view of another non-limiting exemplary embodiment of a piloting element for a rotational atherectomy device;

FIG. 7 is a side view of the piloting element of FIG. 6;

FIG. 8 is an end view of the piloting element of FIGS. 6-7 from a distal end thereof;

FIG. 9 is an end view of the piloting element of FIGS. 6-8 from a proximal end thereof;

FIG. 10 is a perspective view of another non-limiting exemplary embodiment of a rotational atherectomy device;

FIG. 11 is a perspective view of yet another non-limiting exemplary embodiment of a rotational atherectomy device;

FIG. 12 is a perspective view of a non-limiting exemplary embodiment of a rotational atherectomy device;

FIG. 13 is a perspective view of the piloting element of FIG. 2 used with the rotational atherectomy device of FIGS. 11 and 12;

FIG. 14 is a side view of the piloting element of FIG. 13;

FIG. 15 is a perspective view of the piloting element of FIG. 6 used with the rotational atherectomy device of FIGS. 11 and 12;

FIG. 16 is a side view of the piloting element of FIG. 15;

FIG. 17 is a perspective view of the piloting element of FIG. 2 used with the rotational atherectomy device of FIGS. 11 and 12 having a flexible drive shaft extending into at least a portion of the piloting element;

FIG. 18 is a side view of the piloting element of FIG. 17;

FIG. 19 is a perspective view of the piloting element of FIG. 6 used with the rotational atherectomy device of FIGS. 11 and 12 having a flexible drive extending through the piloting element; and

FIG. 20 is a side view of the piloting element of FIG. 19.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Various embodiments of the present invention comprise a rotational atherectomy system as described generally in U.S. Pat. No. 6,494,890, entitled “ECCENTRIC ROTATIONAL ATHERECTOMY DEVICE,” which is incorporated herein by reference. Additionally, the disclosure of the following co-owned patents or patent applications are herein incorporated by reference in their entireties: U.S. Pat. No. 6,295,712, entitled “ROTATIONAL ATHERECTOMY DEVICE”; U.S. Pat. No. 6,132,444, entitled “ECCENTRIC DRIVE SHAFT FOR ATHERECTOMY DEVICE AND METHOD FOR MANUFACTURE”; U.S. Pat. No. 6,638,288, entitled “ECCENTRIC DRIVE SHAFT FOR ATHERECTOMY DEVICE AND METHOD FOR MANUFACTURE”; U.S. Pat. No. 5,314,438, entitled “ABRASIVE DRIVE SHAFT DEVICE FOR ROTATIONAL ATHERECTOMY”; U.S. Pat. No. 6,217,595, entitled “ROTATIONAL ATHERECTOMY DEVICE”; U.S. Pat. No. 5,554,163, entitled “ATHERECTOMY DEVICE”; U.S. Pat. No. 7,507,245, entitled “ROTATIONAL ANGIOPLASTY DEVICE WITH ABRASIVE CROWN”; U.S. Pat. No. 6,129,734, entitled “ROTATIONAL ATHERECTOMY DEVICE WITH RADIALLY EXPANDABLE PRIME MOVER COUPLING”; U.S. Pat. No. 8,597,313, entitled “ECCENTRIC ABRADING HEAD FOR HIGH-SPEED ROTATIONAL ATHERECTOMY DEVICES”; U.S. Pat. No. 8,439,937, entitled “SYSTEM, APPARATUS AND METHOD FOR OPENING AN OCCLUDED LESION”; U.S. Pat. Pub. No. 2009/0299392, entitled “ECCENTRIC ABRADING ELEMENT FOR HIGH-SPEED ROTATIONAL ATHERECTOMY DEVICES”; U.S. Pat. Pub. No. 2010/0198239, entitled “MULTI-MATERIAL ABRADING HEAD FOR ATHERECTOMY DEVICES HAVING LATERALLY DISPLACED CENTER OF MASS”; U.S. Pat. Pub. No. 2010/0036402, entitled “ROTATIONAL ATHERECTOMY DEVICE WITH PRE-CURVED DRIVE SHAFT”; U.S. Pat. Pub. No. 2009/0299391, entitled “ECCENTRIC ABRADING AND CUTTING HEAD FOR HIGH-SPEED ROTATIONAL ATHERECTOMY DEVICES”; U.S. Pat. Pub. No. 2010/0100110, entitled “ECCENTRIC ABRADING AND CUTTING HEAD FOR HIGH-SPEED ROTATIONAL ATHERECTOMY DEVICES”; U.S. Design Pat. No. D610258, entitled “ROTATIONAL ATHERECTOMY ABRASIVE CROWN”; U.S. Design Pat. No. D6107102, entitled “ROTATIONAL ATHERECTOMY ABRASIVE CROWN”; U.S. Pat. Pub. No. 2009/0306689, entitled “BIDIRECTIONAL EXPANDABLE HEAD FOR ROTATIONAL ATHERECTOMY DEVICE”; U.S. Pat. Pub. No. 2010/0211088, entitled “ROTATIONAL ATHERECTOMY SEGMENTED ABRADING HEAD AND METHOD TO IMPROVE ABRADING EFFICIENCY”; and U.S. Pat. Pub. No. 2013/0018398, entitled “ROTATIONAL ATHERECTOMY DEVICE WITH ELECTRIC MOTOR.”

In addition to the foregoing, or in the alternative, co-owned U.S. Pat. No. 8,348,965, titled “ROTATIONAL ATHERECTOMY DEVICE WITH COUNTERWEIGHTING”, which is hereby incorporated by reference in its entirety, discloses non-limiting exemplary embodiments of rotational atherectomy devices having a flexible, elongated, rotatable drive shaft with an abrasive section comprising an enlarged diameter section of the drive shaft or, alternatively, a solid abrasive crown which may be attached to the drive shaft. The device further comprises a proximal and/or a distal counterweight attached to the drive shaft, spaced from the abrasive section wherein each counterweight has its center of mass offset from the longitudinal axis of the drive shaft to stimulate orbital motion by the abrasive section. When placed within an artery against stenotic tissue and rotated at sufficiently high speeds (e.g., in the range of about 20,000 rpm to about 200,000 rpm) the orbiting nature of the abrasive section causes such section to rotate as to open the stenotic lesion to a diameter substantially larger than the resting outer diameter of the abrasive section.

In further addition to the foregoing, co-owned U.S. Pat. Nos. 8,551,128 and 8,628,551, titled “ROTATIONAL ATHERECTOMY DEVICE WITH PRE-CURVED DRIVE SHAFT”, which are hereby incorporated by reference in their entirety, disclose a rotational atherectomy system, device and method comprising a flexible, elongated, rotatable drive shaft with an abrasive section within a pre-curved section of the drive shaft. The device may further comprise a concentric or eccentric enlarged diameter section that is at least partially covered with abrasive material to comprise the abrasive section. The abrasive section may further comprise an abrasive crown or burr mounted to the drive shaft. The pre-curved drive shaft allows smaller diameter and/or massive abrasive regions to be used while sweeping larger diameters during high-speed rotation. The pre-curved region is substantially straightened for insertion into vasculature and placement adjacent stenosis by insertion of the guide wire. Removal of guide wire proximally from the pre-curved region allows the drive shaft to return to its pre-curved form for ablation. Reinsertion of the guide wire beyond the pre-curved region straightens the drive shaft for ease of removal.

It is contemplated that one or more features, including configurations, placement, location, operational and functional characteristics, etc., of the various non-limiting exemplary embodiments of any and all abrading elements are equally or substantially equally applicable for the piloting elements of the instant disclosure. One or more such piloting elements may be provided individually and/or in combination with one or more abrading elements.

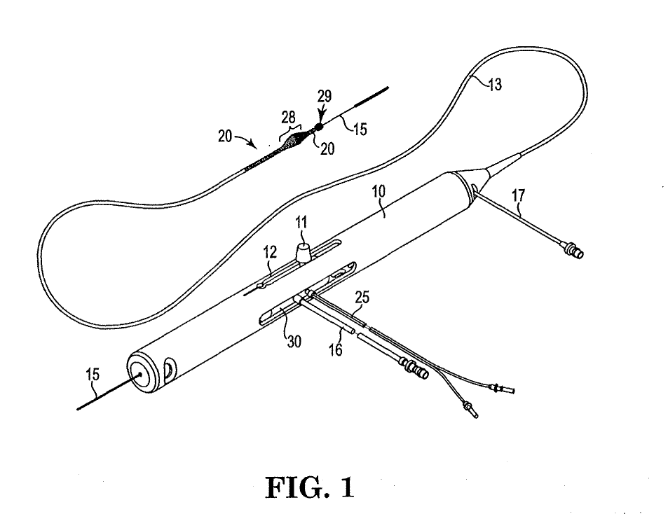

FIG. 1 illustrates one embodiment of a rotational atherectomy device according to the present invention. The device includes a handle portion 10; an elongated, flexible drive shaft 20 having an eccentric abrading element 28 and a piloting element 29 comprising either a piloting tip or bushing mounted or otherwise disposed on the flexible drive shaft at a point distal to the abrading element 28; and an elongated catheter 13 extending distally from the handle portion 10. The drive shaft 20 is constructed from helically coiled wire as is known in the art and the abrading element 28 and the piloting element 29 are fixedly attached to the drive shaft 20. The drive shaft 20 has an outer surface 24 and an inner surface 22 defining an inner lumen, permitting the drive shaft 20 to be advanced and rotated over a guide wire 15. The catheter 13 has a lumen in which most of the length of the drive shaft 20 is disposed, except for the enlarged abrading element 28 and a section of the drive shaft 20 distal to the enlarged abrading element 28. A fluid supply line 17 may be provided for introducing a cooling and lubricating solution (typically saline or another biocompatible fluid) into the catheter 13.

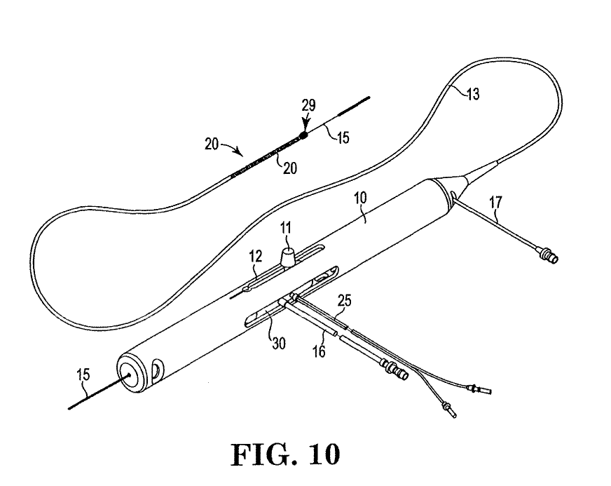

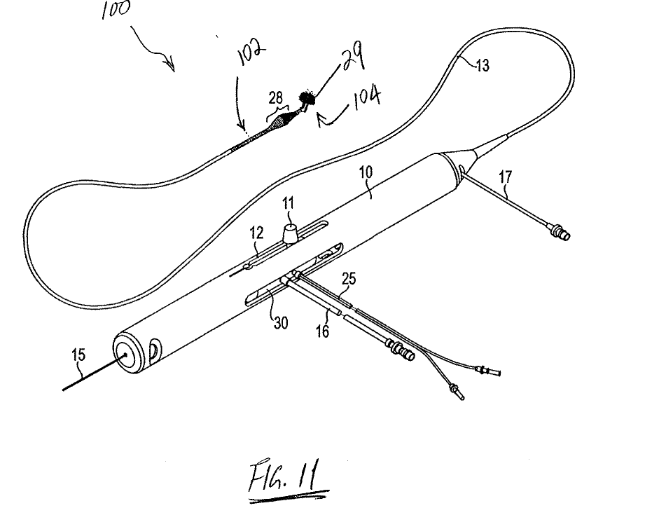

FIG. 10 illustrates another non-limiting exemplary embodiment of a rotational atherectomy device which does not include the abrading element 28. In all other aspects, the device illustrated in FIG. 10 is substantially similar to that described with reference to FIG. 1.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20 (details regarding such handles and associated instrumentation are well known in the industry, and are described, e.g., in U.S. Pat. No. 5,314,407, issued to Auth). The handle 10 also desirably includes a control knob 11 for advancing and retracting the turbine and drive shaft 20 with respect to the catheter 13 and the body of the handle.

As discussed above, in at least one embodiment, the eccentric abrading element 28 comprises an eccentric enlarged section of the drive shaft, or an eccentric solid crown, or an eccentric burr attached to the drive shaft. In some embodiments, the abrading element 28 has a center of mass spaced radially from the rotational axis of the drive shaft 20, facilitating the ability of the device to open the stenotic lesion to a diameter substantially larger than the outer diameter of the abrading element 28. This may be achieved by spacing the geometric center of the abrading element 28, i.e., the eccentric enlarged diameter section of the drive shaft 20, or the eccentric solid abrading element, e.g., an abrading head or abrading crown or abrading burr, attached to the drive shaft 20, away from the rotational axis of the drive shaft 20. Alternatively, the center of mass of the abrading element 28 may be radially spaced from the drive shaft's rotational axis by providing an abrading element 28 that comprises a differential combination of materials, wherein one side of at least one of the abrading element 28 comprises a more massive or denser material than the other side, which creates eccentricity as defined herein. As those skilled in the art will recognize, creation of eccentricity as by differential use of materials within the structure of the abrading element 28, e.g., a center of mass offset from the drive shaft's rotational axis, is applicable to any embodiment of the abrading element 28 discussed herein, whether concentric, eccentric solid burr, partially hollow crown or abrading element or an enlarged section of the drive shaft, or the equivalent. When rotated at high rotational speeds, the drive shaft 20 stimulates orbital motion of the eccentric abrading element 28 to generate a cutting diameter that is greater than a diameter of the abrading element.

In the present invention, the abrading element 28 may comprise a concentric profile or an eccentric profile. In some embodiments, the abrading element 28 may achieve orbital motion, generated by a positioning of the center of mass of the abrading element 28 radially offset from the rotational axis of the drive shaft, either by using different densities of materials and/or geometrically moving the center of mass of the abrading element 28 radially away from the drive shaft's center of mass. This “eccentricity” may be achieved in either a concentric or an eccentric geometric profile. The abrading element 28 may be an enlarged section of the drive shaft, a burr, or a contoured abrading element and may comprise diamond coating. In other embodiments, the abrading element 28 may comprise a center of mass that is on the drive shaft's rotational axis.

However, these known abrading elements 28 described above are limited to the minimum size lesions that can be treated because the abrasive features of the abrading element are of a diameter that is larger than the drive shaft diameter. The present device remedies that problem, among others. Further, if known abrading elements are forced or driven into a lesion, the abrading element 28 may grip and screw/auger into the lesion with a subsequent building and releasing of force that may undesirably affect the lesion or the blood vessel. The present invention addresses this problem by opening a pilot hole with a diameter equivalent to the diameter of the flexible drive shaft of the atherectomy system. This allows for the minimum required clearance between the abrading element 28 and the lesion to prevent gripping and screwing into the lesion.

The piloting element 29 may be fixedly attached to the drive shaft 20, either by being mounted directly onto the outer surface of the drive shaft or mounted axially to the drive shaft at a distal end of the drive shaft. Since the piloting element 29 is fixedly attached to the drive shaft 20, where the abrading element 28 is also fixedly attached, the piloting element 29 will rotate in the same direction and at the same speed as the abrading element 28.

The piloting element 29 may be coupled to the drive shaft 20 with a concentric or eccentric profile abrading element 28 as described with reference to FIG. 1. In an alternate embodiment, the piloting element 29 may be coupled to the drive shaft 20 without the abrading element 28. The piloting element 29 may be coupled with an abrading element 28 of either a concentric or eccentric geometric profile, wherein the abrading element's center of mass is offset radially from the drive shaft's center of mass. The piloting element 29 may also be coupled with an abrading element 28 of concentric or eccentric geometric profile, wherein the abrading element's enter of mass is collinear with the drive shaft's center of mass. The piloting element 29 coupled to the drive shaft 20 with or without the abrading element 28 may also comprise a concentric or eccentric profile. Irrespective of the presence or absence of the abrading element 28, the piloting element may also comprise a center of mass that is either collinear with the rotational axis of the drive shaft or that is offset radially from the drive shaft's rotational axis using the same techniques discussed above in connection with the abrading element 28. As such, in the absence of the abrading element 28, the piloting element 29 so configured will have operational and functional characteristics similar to those described for the abrading element 28. In at least one embodiment, where the abrading element 28 is eccentric and the piloting element 29 is concentric, the abrading element 28 will act as a counterweight, causing orbital motion of the piloting element 29 and thereby creating an increased rotational diameter for the abrading element 28. In some embodiments, the abrading element 28 and the piloting element 29 are both eccentric and in still other embodiments, the abrading element 28 and the piloting element 29 are both concentric. In a non-limiting exemplary embodiment without the abrading element 28, the eccentricity and/or the positioning of the center of mass of the piloting element 29 may also increase its rotational working diameter.

The piloting element 29 may be spaced apart from the abrading element 28 along the drive shaft 20. In other embodiments, a proximal end of the piloting element 29 abuts a distal end of the abrading element 28. Piloting element 29 in at least some embodiments comprises a distalmost tip that is of the same diameter as the drive shaft to facilitate opening of stenosis in preparation for the abrading element's rotational entry therein.

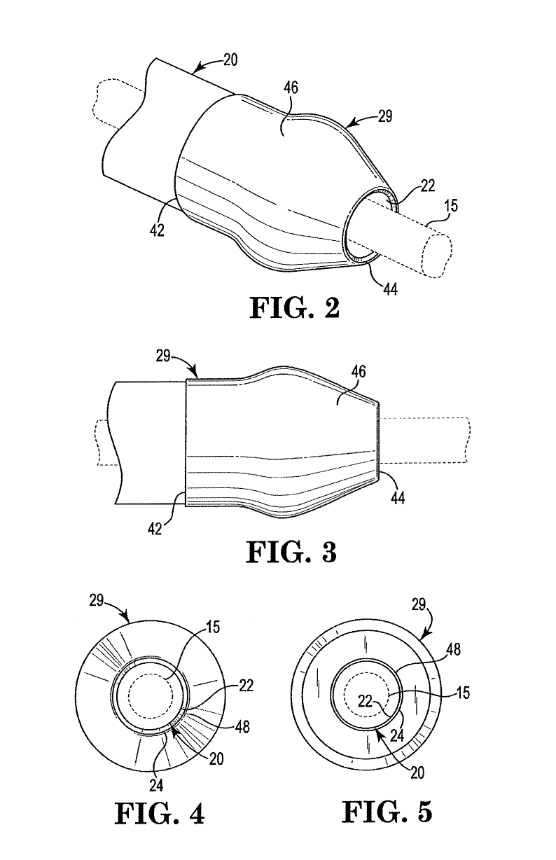

FIGS. 2-9 illustrate some non-limiting exemplary profiles of the piloting element 29. In certain embodiments, the piloting element 29 has a proximal end 42, a distal end 44, an outer surface 46, and an inner surface 48 that defines a lumen. In some embodiments, where the piloting element 29 is fixedly disposed about an outer surface of the drive shaft 20, the inner surface 48 of the piloting element 29 mates or is engaged with the outer surface 24 of the drive shaft 20. In other embodiments, the piloting element 29 may be fixedly attached to a distal end of the drive shaft 20, and the lumen defined by the inner surface 48 allows the piloting element 29 to be advanced and rotated over a guide wire 15. Importantly, the piloting element 29 is fixedly disposed to the outer surface of the drive shaft or fixedly attached to a distal end of the drive shaft 20 such that it rotates simultaneously with the abrading element, rather than separately or selectively rotated.

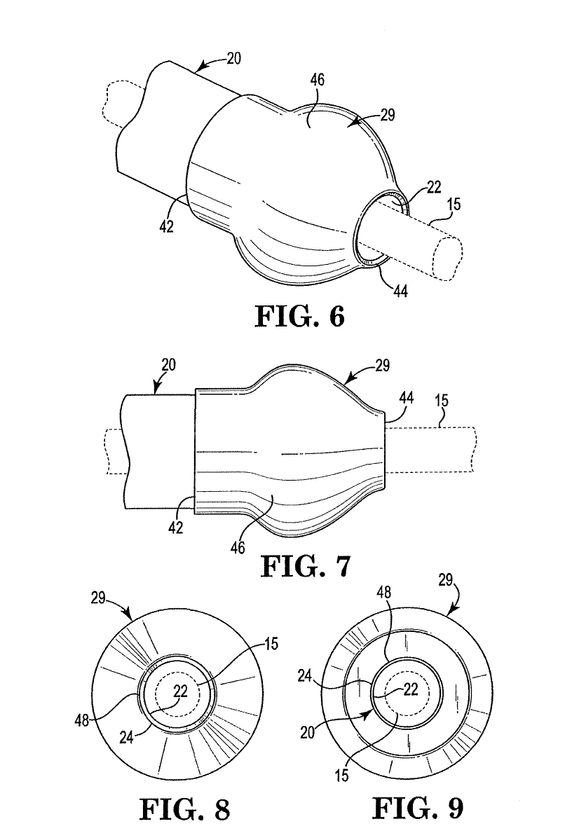

The piloting element 29 may have a shape with a distal end having a diameter smaller than the proximal end. In some embodiments, the piloting element 29 increases in diameter from the distal end 44 to the proximal end 42. In some embodiments, the piloting element 29 has a bulbous profile. In some embodiments, such as the embodiments shown in FIGS. 2-3, the outer diameter of the piloting element 29 has a constant diameter in a proximal section extending distally of the proximal end 42; in an intermediate section, the diameter of the piloting element 29 increases to a maximum point at a distal end of the intermediate section; and in a distal section, the diameter of the piloting element 29 tapers at a constant slope to a diameter at the distal end 44 less than the constant diameter at the proximal end. In some embodiments, such as the embodiment shown in FIGS. 6-7, the outer diameter of the piloting element 29 has a constant diameter in a proximal section extending distally of the proximal end 42; in an intermediate section, the diameter of the piloting element 29 increases to a maximum point at a distal end of the intermediate section; and in a distal section, the outer diameter of the piloting element 29 decreases to a diameter at the distal end 44 less than the constant diameter at the proximal end. In some embodiments, the outer diameter of the piloting element 29 may decrease to a diameter less than the outer diameter of the drive shaft. In the embodiments shown in FIGS. 2-9, the piloting element 29 is symmetrical about a central axis. In other embodiments, the piloting element 29 is asymmetrical about the central axis, such that the piloting element 29 has an orbital path, which may or may not be different than the orbital path of the abrading element 28.

The piloting element 29 may have an abrasive coating disposed on some or all of the outer surface 46 of the piloting element 29. The abrasive coating may be disposed in discrete areas in a desired pattern. In some embodiments, the piloting element 29 has a cutting feature on the outer surface 46. In some embodiments, the piloting element 29 has an impact feature on the outer surface 46. In some embodiments, the piloting element 29 has a thread-like cutting feature disposed about the outer surface 46. In some embodiments, the piloting element 29 is shaped like an auger drill bit with a helical screw blade.

In some embodiments, as will be readily apparent to a person having ordinary skill in the art, the piloting element 29 can also be used for creating a piloting lumen through the stenosis or for creating a cavity extending distally from the piloting hole into the stenosis. For instance, in a non-limiting exemplary embodiment, this can be accomplished by continuing to advance the piloting element 29 distally through the stenosis after the piloting hole is drilled. The piloting lumen can be thus created by the atherectomy device with or without the abrading element 28. For devices having the abrading element 28 proximal of the piloting element 29, the piloting lumen can be created by spacing the abrading element 28 and the piloting element 29 apart by a distance approximately equal to a length of the stenosis. As described elsewhere, in a non-limiting exemplary embodiment, a diameter of the piloting lumen can be made greater than the maximum outer diameter of the piloting element 29 by using a piloting tip or bushing having a center of mass offset radially from a rotational axis, using an eccentric piloting element 29, affixing an element having a mass proximal and/or distal of the piloting element 29 so as to induce an eccentric rotational path. In a non-limiting exemplary embodiment, the abrading element 28 can be used, as described elsewhere, for creating the diameter of the piloting lumen greater than the maximum outer diameter of the piloting element 29. Additional embodiments for configuring and/or using the piloting element 29 for creating a piloting hole in and/or a piloting lumen through a stenosis, as described herein, will become apparent to a person having ordinary skill in the art. All such embodiments are considered as being within the metes and bounds of the instant disclosure as claimed.

A method for opening a stenosis in a blood vessel having a given diameter, comprising: providing a guide wire having a maximum diameter less than the diameter of the artery; advancing the guide wire into a blood vessel to a position proximal to the stenosis; providing a flexible elongated, rotatable drive shaft advanceable over a guide wire, the guide wire having a maximum diameter less than the diameter of the artery; the drive shaft having a rotational axis; the drive shaft having at least one eccentric abrading element 28 and a piloting element 29 fixedly attached to the drive shaft; advancing the piloting element 29 into the artery to a position proximal to the stenosis; creating a piloting hole by rotating the drive shaft at a sufficient rotational speed; advancing the abrading element 28 through the piloting hole, rotating the drive shaft at the rotational speed, and moving the across the stenotic lesion, thereby opening the stenotic lesion to a diameter larger than the nominal diameter of the eccentric enlarged diameter section.

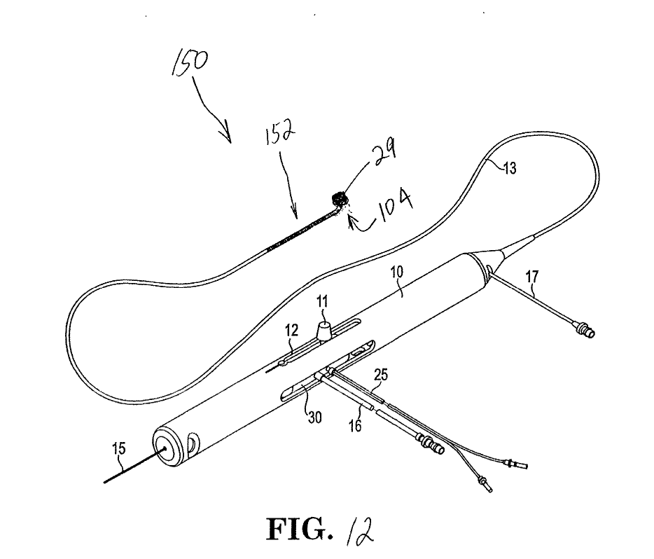

FIGS. 11 and 12, respectively, illustrate non-limiting exemplary embodiments of rotational atherectomy devices 100 and 150 with the guide wire 15 retracted. Devices 100 and 150 are substantially similar to the non-limiting exemplary rotational atherectomy devices described elsewhere with reference to FIGS. 1 and 10. One difference between devices 100 and 150, and the devices of FIGS. 1 and 10 is the draft shaft. While the drive shaft 20 in the devices of FIGS. 1 and 10 is substantially straight throughout its longitudinal extent, devices 100 and 150, respectively, include drive shafts 102 and 152 having a pre-curved or pre-bent distal section 104. As illustrated in FIGS. 11 and 12, the piloting element 29 is fixedly attached to the pre-bent distal section 104 in the manner described elsewhere with reference to FIGS. 1-10. As illustrated in FIG. 11, device 100 includes the abrading element 28 fixedly attached to the substantially straight section of the drive shaft 102 proximal of the pre-bent distal section 104 in the manner described elsewhere with reference to FIGS. 1-10. In contrast to device 100, device 150 does not include the abrading element 28.

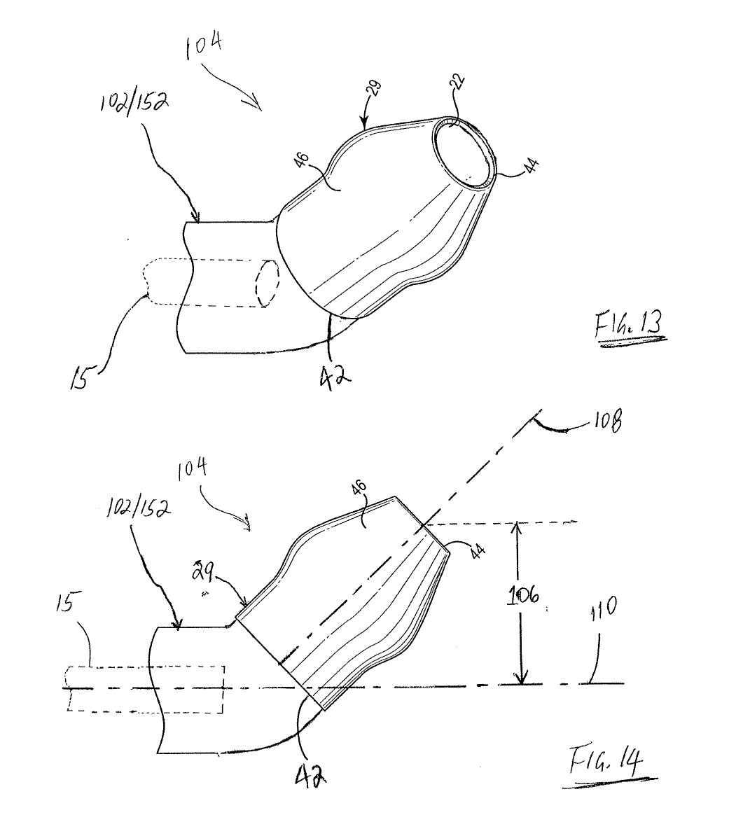

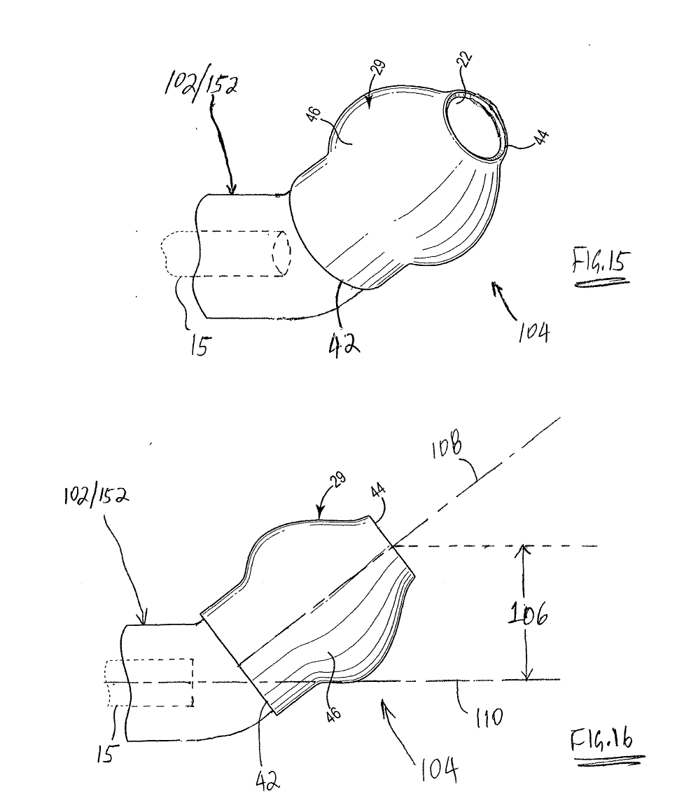

In a non-limiting exemplary embodiment, device 100 is configured such that the guide wire 15 can be used for straightening the pre-bent distal section 104, or for permitting the distal section 104 to bend to its pre-bent profile. For instance, as in the exemplary embodiments illustrated in FIGS. 2, 3, 6 and 7, the distal section 104 can be straightened when the guide wire 15 traverses or extends through the distal section 104. As illustrated in FIGS. 13-16, the distal section 104 returns to its pre-bent profile when the guide wire 15 is retracted proximally such that no portion of the guide wire 15 traverses or extends through the distal section 104. Of course, the piloting element 29, which is fixedly attached to the distal section 104, will also straighten and bend with the distal section 104.

In a non-limiting exemplary embodiment, device 100 may be configured with the distal section 104 (and the piloting element 29) having two distinct or discrete positions, viz., a substantially straight position and a maximally bent position such as illustrated in FIGS. 13-16. For instance, as in the exemplary embodiments illustrated in FIGS. 2, 3, 6 and 7, the distal section 104 may remain substantially straight while any portion of the guide wire 15 traverses or extends therethrough. Then, when a distal end of the guide wire 15 is retracted proximally out of the distal section 104, the distal section 104 becomes fully bent when no portion of the guide wire 15 traverses or extends through the distal section 104 as illustrated in FIGS. 13-16. And, when any portion of the guide wire 15, including its distal end, is extended distally into the bent distal section 104, the entire distal section 104 becomes substantially straight as illustrated in FIGS. 2, 3, 6 and 7. In some embodiments, the guide wire 15 does not have to extend or traverse the entire distal section 104 for straightening it.

In another non-limiting exemplary embodiment, device 100 may be configured with the distal section 104 having a continuously varying bent profile dictated by the extent or length of the guide wire 15 within the distal section 104. For instance, as in the exemplary embodiments illustrated in FIGS. 2, 3, 6 and 7, the distal section 104 may remain substantially straight while the guide wire 15 traverses or extends through the entirety of the distal section 104. Then, as the distal end of the guide wire 15 is retracted proximally through the distal section 104, at least that portion of the distal section 104 distal of the distal end of the guide wire 15 starts bending and reaches its maximum pre-bent profile when no portion of the guide wire 15 traverses or extends through the distal section 104 as illustrated in FIGS. 13-16. And, when the distal end of the guide wire 15 is extended distally into the bent distal section 104, the proximal portion of the distal section 104 traversed by the guide wire 15 proximal of the guide wire's distal end will become substantially straight while the distal portion of the distal section 104 distal of the distal end of the guide wire 15 will be bent. The entire distal section 104 will become substantially straight, as in the exemplary embodiments illustrated in FIGS. 2, 3, 6 and 7, when the guide wire 15 traverses or extends through the entire distal section 104.

As described elsewhere, and as will be well known to a person having ordinary skill in the art, the drive shaft 102/152 extends within the lumen of the catheter 13 and is delivered over the guide wire 15 to the location of the stenosis. Furthermore, the drive shaft 102/152 is configured for rotating about the guide wire 15 extending therethrough. Accordingly, a rotational axis 110 of the drive shaft 102/152 and the longitudinal axis of the guide wire 15 will be substantially coincident with each other. With comparative reference to FIGS. 2, 3, 6 and 7 and FIGS. 13-16, it is seen that when a distal end 112 of the guide wire 15 is retracted proximally out of the distal section 104 such that no portion of the guide wire 15 traverses or extends through the distal section 104, the distal section 104 is maximally bent to its default pre-bent profile as illustrated in FIGS. 13-16. With the guide wire 15 retracted into the lumen of the substantially straight drive shaft 102/152 proximal of the pre-bent distal section 104, the rotational axis 110 of the drive shaft 102/152 and the longitudinal axis of the guide wire 15 will remain substantially coincident with each other. Thus, it is seen that irrespective of the maximally bent or partially bent or straight profile of the distal section 104, the rotational axis 110 of the drive shaft 102/152 and the longitudinal axis of the guide wire 15 remain substantially coincident with each other. As can be seen, the rotational axis 110 of the drive shaft 102/152 and the longitudinal axis of the guide wire 15 remain substantially coincident with each other irrespective of whether or not the guide wire 15, or any portion thereof, traverses or extends through the distal section 104 and irrespective of the profile of the distal section 104. In that which follows, the terms “rotational axis 110 of the drive shaft 102/152” and “longitudinal axis of the guide wire 15” may be used interchangeably, and are both referenced by the numeral 110.

When, as illustrated in FIGS. 13-16, the distal section 104 is maximally bent to its default pre-bent profile, i.e., when no portion of the guide wire 15 traverses or extends through the distal section 104, a distance 106 at the distal end 44 of the piloting element 29 between a longitudinal axis 108 of the distal section 104 and the rotational axis 110 of the drive shaft 102/152 will be a maximum. In other words, piloting element 29, and its distal end 44 in particular, be maximally offset or spaced away from the rotational axis 110 of the drive shaft and the substantially coincident longitudinal axis of the guide wire 15. As described elsewhere, and as will be apparent to a person having ordinary skill in the art, when the drive shaft 102/152 rotates about its rotational axis 110, the piloting element 29, being spaced away from the rotational axis 110, will have an orbital path which will also be offset or spaced away from the rotational axis 110 of the drive shaft 102/152. In some embodiments having a concentric piloting element 29 with a center of mass and/or geometrical center coincident with the longitudinal axis 108, the diameter of the orbital path, i.e., the distance between the orbital path and the rotational axis 110, will be approximately the same as or slightly larger than the distance 106. In certain embodiments, depending on the shape of the piloting element 29, the “slightly larger” diameter may be approximately equal to the sum of the distance 106 and the maximum distance between the outer surface 46 and the longitudinal axis 108 of the distal section 104. In some embodiments having an eccentric piloting element 29 and/or having a center of mass and/or geometrical center not coincident with the longitudinal axis 108, the diameter of the orbital path, i.e., the distance between the orbital path and the rotational axis 110, will be a function of the eccentricity of the piloting element 29 and/or the location of the center of mass and/or the geometrical center. In certain embodiments, the diameter of the orbital path will not be a constant, but will change as the piloting element orbits or rotates about the rotational axis 110 of the drive shaft 102/152. As such, the diameter of the orbital path at the numerous circumferential locations about the rotational axis 110 may be less than and/or equal to and/or greater than the distance 106.

In a non-limiting exemplary embodiment, the distance 106 will depend on the default pre-bent profile of the distal section 104. In another non-limiting exemplary embodiment, the distance 106 can be customized and/or changed prior to using the device 100. For instance, the device 100 may be manufactured and supplied with a substantially straight distal section 104 or with the distal section 104 having a default pre-set bent profile, and the distal section 104 can be bent to a default pre-bent profile as desired by the user. In a non-limiting exemplary embodiment, a shape-memory material may be used at least in that section or location of the drive shaft 102/152 where the bend will be made.

With reference to FIGS. 2, 3, 6 and 7, it is seen that when the guide wire 15 traverses or extends through the distal section 104, the distal section 104 will be substantially straight and substantially aligned with the drive shaft 102/152 proximal of the distal section 104. As such, the longitudinal axis 108 of the distal section 104 and the rotational axis 110 of the drive shaft 102/152 will be substantially coincident with each other, and the distance 106 will be negligibly small. As described elsewhere, the orbital path and/or the diameter of the orbital path of the piloting element 29 will be a function of one or more factors pertaining to the piloting element 29, including the eccentricity or concentricity of the piloting element 29, the location of the center of mass and/or the geometrical center of the piloting element 28, the shape of the piloting element 29, the presence or absence of the abrading element 28, etc. If the drive shaft 102/152 includes the abrading element 28, the orbital path and/or the diameter of the orbital path of the piloting element will also be a function of one or more factors pertaining to the abrading element 28, including the eccentricity or concentricity of the abrading element 28, the location of the center of mass and/or the geometrical center of the abrading element 28, the shape of the abrading element 28, etc.

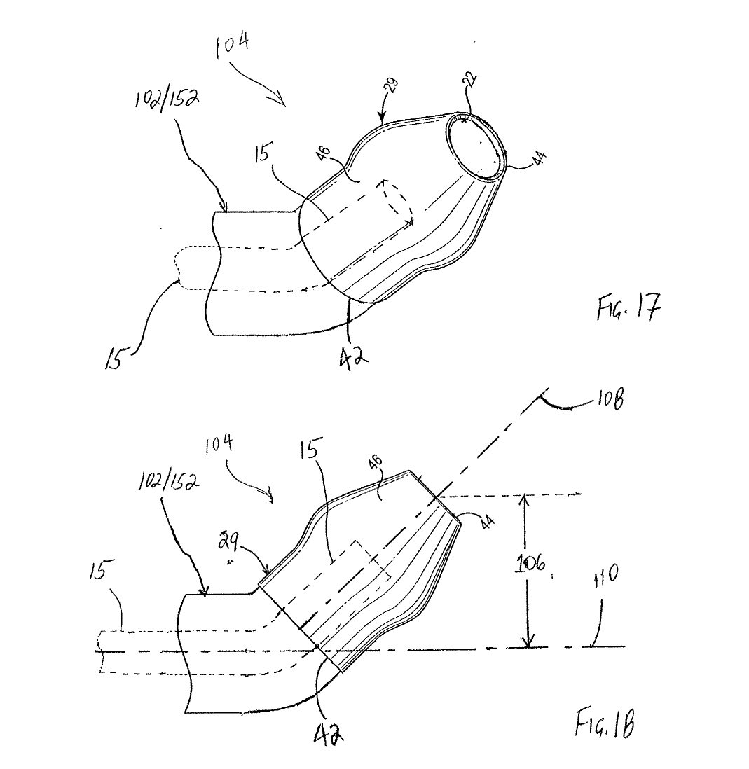

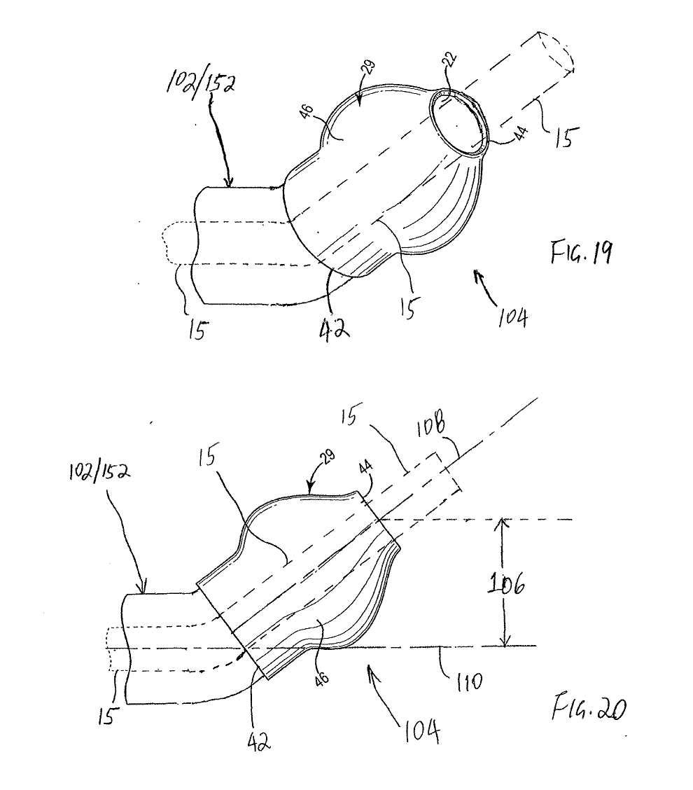

In a non-limiting exemplary embodiment, the fully bent, partially bent or straight profile of the distal section 104 may be affected by retracting or extracting the distal section 104 into or out of the catheter 13. For instance, if no portion of the guide wire 15 traverses or extends through the distal section 104 and/or if the guide wire 15 extending through or at least into a portion of the distal section 104 is sufficiently flexible, then the catheter 13 may be used for fully or partially straightening the pre-bent distal section 104 by retracting the entire or a portion of the distal section 104 proximally into the lumen of the catheter 13 through which the drive shaft 102/152 extends. Conversely, extracting or extending the entire or a portion of the distal section 104 in the distal direction out of the lumen of the catheter 13 will fully or partially bend at least that portion of the pre-bent distal section 104 that has been extracted out of the lumen of the catheter 13. Of course, a combination of the catheter 13 and the guide wire 15 may be used for fully or partially straightening and/or bending the entire and/or a portion of the piloting element 29. Non-limiting exemplary embodiments of rotational atherectomy devices having a flexible drive shaft 15 extending into a portion of or through the distal section 104, such those described, are illustrated in FIGS. 17-20 wherein, FIGS. 17 and 18, respectively, are a perspective view and a side view of the distal section 104, i.e., the piloting element, illustrated with the flexible drive shaft 15 extending into at least a portion thereof; and FIGS. 19 and 20, respectively, are a perspective view and a side view of the distal section 104, i.e., the piloting element, illustrated with the flexible drive shaft 15 extending therethrough.

In some embodiments, the catheter 13 may be used for affecting the profile (i.e., bent, straight, and/or partially bent/straight) while the guide wire 15 traverses or extends through the entire distal section 104 or through at least a portion of the distal section 104. In other words, such embodiments do not require the guide wire 15 to be completely retracted out of the distal section 104 before retracting or extracting the distal section 104 into or out of the catheter 13. In certain embodiments, the guide wire 15 must be completely retracted out of the distal section 104 before retracting or extracting the distal section 104 into or out of the catheter 13. In other words, the guide wire 15 must be completely extracted out of the distal section 104 before the catheter 13 may be used for affecting the profile (i.e., bent, straight, and/or partially bent/straight) of the distal section 104. Accordingly, in some embodiments, the guide wire 15 may be highly flexible. In certain embodiments, the guide wire 15 may be sufficiently stiff such that it bends or straightens out with the distal section 104 while at the same time being sufficiently flexible for traversing the vasculature.

In a non-limiting exemplary embodiment wherein the distal section 104 (and the piloting element 29) has a continuously varying bent profile, the distance 106 will increase or decrease as determined by the extent of the guide wire 15 traversing or extending through the distal section 104. For instance, the distance 106 will increase as the distal end of the guide wire 15 is retracted proximally through the distal section 104, and the distance 106 will decrease as the distal end of the guide wire 15 is extended distally through the distal section 104. As described elsewhere, the distance 106 will be a maximum when the distal section 104 is devoid of any portion of the guide wire 15, i.e., when the distal end of the guide wire 15 has been retracted proximal of the distal section 104.

When the guide wire 15 extends or traverses through the entire distal section 104, the entire drive shaft 102/152, including the pre-bent distal section 104 having the piloting element 29, will be substantially straight, and the rotational axis 110 of the drive shaft 102/152, including the pre-bent distal section 104 having the piloting element 29, and the longitudinal axis of the guide wire 15 will be substantially coincident with each other. As such, the entirety of the substantially straight drive shaft 102/152 will rotate about the longitudinal axis of the guide wire 15, and the piloting element 29 will have an orbital path as described elsewhere with reference to FIGS. 1 and 10. When a portion of or the entire distal section 104 is bent, the rotational axis 110 of the drive shaft 102/152 and the longitudinal axis of the guide wire 15 will be substantially coincident with each other. The piloting element 29 affixed on the bent distal section 104 will be radially offset or spaced apart from the substantially coincident rotational axis 110 of the drive shaft 102/152 and the longitudinal axis of the guide wire 15. Accordingly, when the drive shaft 102/152 rotates about its rotational axis 110, i.e., about the longitudinal axis of the guide wire 15, the piloting element 29 will have an orbital path that is radially spaced away or offset from the rotational axis 110 of the drive shaft 102/152, and the diameter of the orbital path traversed by the piloting element 29 on the bent distal section 104 will be larger than the diameter of the orbital path traversed by the piloting element 29 on a substantially straight, i.e., unbent, distal section 104. In a non-limiting exemplary embodiment, the diameter of the orbital path will be substantially the same as the distance 106. In another non-limiting exemplary embodiment, the diameter of the orbital path will depend on several parameters, including the distance 106 and the configuration and/or the structure of the piloting element 29 such as its eccentricity, location of its center of mass, location of its geometrical center, etc. The operational characteristics and functionality of different configurations and/or structures of the piloting element 29, and their effect on its orbital path, are described elsewhere with reference to FIGS. 1-9.

In some non-limiting exemplary embodiments, the abrading element 28 and the piloting element 29 are configured, i.e., constructed, and attached to the drive shaft 20/102/152 such that their orbital paths are substantially parallel to each other. In certain embodiments, the orbital paths are not parallel to each other. In some embodiments, the diameters of the orbital paths of the abrading element 28 and the piloting element 29 are substantially the same. In certain embodiments, the orbital paths of the abrading element 28 and the piloting element 29 have different diameters. In some embodiments, the center of mass and/or the geometrical center of the abrading element 28 and of the piloting element 29 are substantially co-linear or not co-linear or substantially co-planar or not co-planar with each other. In certain embodiments, the center of mass and/or the geometrical center of the abrading element 28 and of the piloting element 29 are angularly offset from each other. For instance, the angular offset of the centers may range from 0° to 360° relative to one another.

In some non-limiting exemplary embodiments, the rotational atherectomy device(s) includes a proximal and/or distal counter-weight for either one or both of the abrading element 28 and the piloting element 29. Non-limiting exemplary embodiments of one or more counter-weights are disclosed in co-owned U.S. Pat. No. 8,348,965, which is incorporated herein by reference in its entirety. While only counter-weights for or associated or coupled with an abrading element are illustrated and described in U.S. Pat. No. 8,348,965, similar or different counter-weights for or associated or coupled with a piloting element are contemplated and are therefore considered as being within the metes and bounds of the instant disclosure. In certain embodiments, one or more counter-weights are included as distinct or discrete components or elements separate from their respective or corresponding abrading element 28 and/or piloting element 29. As such, the one or more counter-weights may be fixedly attached to or otherwise disposed on the drive shaft either proximate to or spaced away from the abrading element 28 and/or the piloting element 29. In some embodiments, one or more counter-weights are integral with or otherwise disposed on their respective or corresponding abrading element 28 and/or piloting element 29. In certain embodiments, any two or more counter-weights may be substantially co-linear or not co-linear or substantially co-planar or not co-planar with each other. In some embodiments, any two-or more counter-weights may be angularly offset from each other at angle(s) ranging between 0° and 360° relative to one another.

In view thereof, it should be readily and clearly apparent to a person having ordinary skill in the art that all configurations, operational and functional characteristics, etc., of an abrading element with or without one or more counter-weights are equally or substantially equally applicable for a piloting element. Furthermore, one or more such piloting elements may be provided individually by themselves or in combination with one or more abrading elements. In addition thereto, one or more such piloting elements may be affixed to or otherwise disposed on a pre-bent or a straight, i.e., not pre-bent, distal section or distal tip or end of the drive shaft. All such embodiments, including modifications thereof, are considered as being within the metes and bounds of the instant disclosure.

Co-owned U.S. Pat. Nos. 8,551,128 and 8,628,551, titled “ROTATIONAL ATHERECTOMY DEVICE WITH PRE-CURVED DRIVE SHAFT”, which are hereby incorporated by reference in their entirety, disclose a variety of techniques for fixedly forming or adapting the pre-bent distal section 104. In a non-limiting exemplary embodiment, a unique heat setting method is used with conventional metal such as stainless steel. Briefly, the method for forming the pre-curved distal section 104 starts with using a coil winder to wind the drive shaft, and then heating the entire length of the wound drive shaft at a pre-determined temperature for a pre-determined duration of time for relaxing and stabilizing the coil dimensions. Next, a mandrel shaped in the desired curved drive shaft form is inserted into the lumen at the distal end of the straight (and pre-relaxed) drive shaft. Thus, the distal section of the drive shaft is forced to take on the shape of the mandrel. Then, with the mandrel in place, a local heat treatment at a pre-determined temperature for a pre-determined duration of time is performed on the curved portion of the drive shaft. After the local heat treatment is complete, the mandrel is removed and the curved shape is retained by the drive shaft thus forming the pre-bent or pre-curved distal section 104.

Other mechanisms and methods for forming the pre-curved distal section 104 may include using shape memory alloy materials. In some non-limiting exemplary embodiments, shape memory alloy materials such as Nitinol, which exhibits super-elastic properties and increased flexibility is used. Additional non-limiting examples of super-elastic metal alloys that are usable for forming the pre-bent or pre-curved distal section 104 are described in detail in U.S. Pat. No. 4,665,906. The disclosure of U.S. Pat. No. 4,665,906 is herein expressly incorporated by reference insofar as it describes the compositions, properties, chemistries, and behavior of specific metal alloys which are superelastic within the temperature range at which the pre-curved distal section 104 of the drive shaft 102/152 operates. Any and all such superelastic metal alloys may be used to form the pre-curved section 104 of the drive shaft 102/152.

Prior to insertion into the vasculature, drive shafts 102/152 having the pre-curved distal section 104 are provided in the pre-curved configuration. The pre-curved distal section 104 is then mechanically “deformed” to a generally linear and/or straight configuration and profile by inserting a substantially linear guide wire 15 into the drive shaft lumen and through the distal section 104. After the combination of the guide wire 15 and the drive shaft 102/152 with the generally linear and/or straight distal section 104, inter alia, has been introduced into the vasculature and the distal end or tip of the drive shaft 102/152 is positioned proximate the target, the guide wire 15 may be retracted proximally thereby allowing the pre-curved distal section 104 to return to its original pre-curved profile and configuration.

Any and all of the above combinations of piloting elements and the abrading elements in rotational atherectomy system are within the scope of the present invention. The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A device, comprising:
a guide wire;
an elongated flexible drive shaft advanceable over the guide wire, the drive shaft comprising:
a pre-bent distal section extending proximally from a distal end of the drive shaft, wherein:
at least that portion of the distal section not traversed by the guide wire comprises a curvilinear profile; and
at least that portion of the distal section traversed by the guide wire comprises a substantially linear profile;
a piloting element fixedly attached to at least a portion of the distal section; and
an abrading element fixedly attached to the drive shaft proximal of the distal section.

2. The device of claim 1, wherein
the piloting element is concentric or eccentric; and
the abrading element is concentric or eccentric.

3. The device of claim 1, wherein:
the piloting element comprises a center of mass collinear with or coplanar with or radially offset from a longitudinal axis of the distal section; and
the abrading element comprises a center of mass collinear with or coplanar with or radially offset from a rotational axis of the drive shaft.

4. The device of claim 1, wherein:
the piloting element is symmetrical or asymmetrical about a longitudinal axis of the distal section; and
the abrading element is symmetrical or asymmetrical about a rotational axis of the drive shaft.

5. The device of claim 1, wherein at least a portion of an outer surface of the piloting element comprises one of:
an abrasive coating;
a cutting feature;
an impact feature; and
an auger.

6. The device of claim 1, wherein the piloting element comprises a bulbous profile.

7. The device of claim 1, wherein the piloting element comprises:
a proximal section extending distally from a proximal end of the piloting element;
a distal section extending proximally from a distal end of the piloting element; and
an intermediate section extending between the proximal and the distal sections of the piloting element.

8. The device of claim 7, wherein the proximal section of the piloting element comprises a substantially constant diameter.

9. The device of claim 7, wherein a diameter of the distal section of the piloting element increases proximally from the distal end of the piloting element.

10. The device of claim 7, wherein a diameter at the distal end of the piloting element is less than a diameter at the proximal end of the piloting element or is less than a diameter of the drive shaft.

11. The device of claim 7, wherein the intermediate section of the piloting element comprises a generally parabolic profile wherein a diameter of the intermediate section of the piloting element increases distally from the proximal section of the piloting element to a maximum diameter and thereafter decreases distally to the distal section of the piloting element.

12. The device of claim 7, wherein a diameter of the intermediate section of the piloting element increases distally from the proximal section of the piloting element to a maximum diameter at a distal end of the intermediate section of the piloting element.

13. The device of claim 1, wherein a distance between a longitudinal axis of the distal section and a longitudinal axis of the guide wire increases as a distal end of the guide wire is retracted proximally from the distal end of the drive shaft through the distal section.

14. The device of claim 13, wherein the distance between the longitudinal axis of the distal section and the longitudinal axis of the guide wire is a maximum when the distal section is not traversed by the guide wire.

15. The device of claim 1, wherein a distance between a longitudinal axis of the distal section and a longitudinal axis of the guide wire decreases as a distal end of the guide wire is extended distally through the distal section.

16. The device of claim 15, wherein within at least that portion of the distal section traversed by the guide wire, the rotational axis of the drive shaft and the longitudinal axis of the guide wire are substantially coincident with each other.

17. The device of claim 1, wherein the piloting element, when rotated, comprises an orbital path offset from a rotational axis of the drive shaft.

18. The device of claim 1, wherein the abrading element comprises:
a proximal portion extending distally from a proximal end of the abrading element;
a distal portion extending proximally from a distal end of the abrading element; and
an intermediate portion extending between the proximal and the distal portions of the abrading element.

19. The device of claim 18, wherein:
a diameter the proximal portion increases distally; and
a diameter the distal portion increases proximally.

20. The device of claim 18, wherein at least a portion of an outer surface of the abrading element comprises one of:
an abrasive coating;
a cutting feature;
an impact feature; and
an auger.

21. The device of claim 1, wherein the abrading element, when rotated, comprises an orbital path having a diameter greater than a maximum diameter of the abrading element.

22. The device of claim 1, comprising a catheter having a lumen extending therethrough, wherein:
the drive shaft extends through the lumen of the catheter; and
the profile of the distal section is affected by a position of the distal section relative to an opening in a distal end of the lumen of the catheter.

23. The device of claim 22, wherein:
the profile of the distal section changes from the substantially linear profile to the curvilinear profile when the entire distal section is extended out of the lumen of the catheter; and
the profile of the distal section changes from the curvilinear profile to the substantially linear profile when the entire distal section is retracted into the lumen of the catheter.

24. The device of claim 23, wherein:
the profile of at least that portion of the distal section extending out of the lumen of the catheter changes from the substantially linear profile to a non-linear profile; and
the profile of at least that portion of the distal section retracted into the lumen of the catheter changes from the non-linear profile to the substantially linear profile.

25. A device, comprising:
a guide wire; and
an elongated flexible drive shaft advanceable over the guide wire, the drive shaft comprising:
a pre-bent distal section extending proximally from a distal end of the drive shaft, wherein:
at least that portion of the distal section not traversed by the guide wire comprises a curvilinear profile; and
at least that portion of the distal section traversed by the guide wire comprises a substantially linear profile; and
a piloting element fixedly attached to at least a portion of the distal section.

26. A device, comprising:
a guide wire; and
an elongated flexible drive shaft advanceable over the guide wire, the drive shaft comprising:
a pre-bent distal section extending proximally from a distal end of the drive shaft, the distal section configured for changing its profile between a pre-determined curvilinear profile and a substantially linear profile;
a piloting element fixedly attached to at least a portion of the distal section; and
an abrading element fixedly attached to the drive shaft proximal of the distal section;
wherein, the guide wire is sufficiently flexible for changing its profile between a curvilinear profile and a substantially linear profile responsive to a change in the profile of the distal section.

27. A device, comprising:
a guide wire; and
an elongated flexible drive shaft advanceable over the guide wire, the drive shaft comprising:
a pre-bent distal section extending proximally from a distal end of the drive shaft, the distal section configured for changing its profile between a curvilinear profile and a substantially linear profile; and
a piloting element fixedly attached to at least a portion of the distal section;
wherein, the guide wire is sufficiently flexible for changing its profile between a curvilinear profile and a substantially linear profile responsive to a change in the profile of the distal section.
